(19) United States
(12) Patent Application Publication
(10) Pub. No.: US 2012/0022543 A1
(43) Pub. Date: Jan. 26, 2012

(54) SYSTEM, METHOD, AND APPARATUS FOR LOCATING A FEMORAL NECK GUIDE WIRE

(75) Inventors: Alec Porzel, Collierville, TN (US); Robert Lowrie, Hernando, MS (US)

(73) Assignee: SMITH & NEPHEW, INC., Memphis, TN (US)

(21) Appl. No.: 13/254,641

(22) PCT Filed: Mar. 5, 2010
(86) PCT No.: PCT/US10/26418

§ 371 Date: Oct. 7, 2011

Related U.S. Application Data
(60) Provisional application No. 61/157,829, filed on Mar. 5, 2009.

(51) Int. Cl. A61B 17/56

(52) U.S. Cl. 606/96

(57) ABSTRACT

A pin alignment guide may be placed on an end of a bone for guiding a pin along a central axis of the bone. The bone has a length extending away from the end of the bone. A body may be configured to overlie the end of the bone. The body may have a contact surface configured to contact the bone and a guide surface configured to guide a pin into the bone. A rod portion may be configured to extend along the length of the bone away from the end of the bone. An extension may be configured to attach to the body and extend away from the body, the extension further configured to couple to the rod portion, such that the rod portion may be aligned along the length of the bone thereby positioning the guide surface of the body to guide the pin.

### CROSS-REFERENCE TO RELATED APPLICATIONS

This application claims priority to U.S. Provisional Application No. 61/157,829 file Mar. 5, 2009. The disclosure of that application is hereby incorparated by reference herein.

### BACKGROUND

1. Field

The present invention relates generally to a system, method, and apparatus for use during hip resurfacing arthroplasty procedures, and more particularly to an apparatus for locating a femoral neck guide wire.

2. Related Art

Smith & Nephew's current Birmingham Hip Resurfacing (BHR) surgical techniques use an X-ray to pre-operatively template the position of a femoral alignment pin. A ruler is used to mark the greater trochanter on the template. Finally, the front and back of the femoral shaft are felt. Such probing of the front and back portions of the femoral shaft allows the center (i.e., medial/lateral midline) of the femoral shaft to be approximated prior to transverse drilling. Drilling originates from the femoral shaft, propagates through the femoral neck, and then finally out the femoral head. After drilling has been completed, another procedure involving the McMinn alignment guide is used to place a guide wire which serves to guide a sleeve cutter.

To this end, the McMinn alignment guide uses a two-sided clamp around the femoral neck to centralize the guide wire through the femoral neck. Based on the variable geometry of the referenced bone, this process at best generally only finds an “approximate” center point in the femoral neck, and an “approximate” center axis.

The step of positioning of a guide wire is crucial to the efficacy of primary hip resurfacing arthroplasty, because the guide wire position and location will ultimately dictate the final position and orientation of the resurfacing implant with respect to femoral bone. The bulkiness and complexity of conventional instruments make the step of position the guide wire very difficult.

The methods and apparatus of the present invention enable a surgeon to accurately find a center axis along the entire femoral neck and accurately locate a guide wire. Instrumentation provided herein is deemed to be substantially less invasive than prior art methods, because it can be utilized with only minimal exposure of the surgical site, and requires less resection of the femoral head (i.e., is more bone-conserving). For example, only the femoral head and neck to be exposed, thus eliminating the need to drill through the distal end of the femoral neck by way of the femoral shaft as previously discussed. Finally, the present invention provides a safe and easy method for finding the center of the femoral neck. Solving these issues will provide maximum strength and residual integrity of the arthroplasty.

### SUMMARY

The aforementioned needs are satisfied by several aspects of the present invention.

According to one aspect of the invention, there is provided a system and method of positioning a guide wire during hip resurfacing arthroplasty procedures.

According to another aspect of the invention, there is provided an apparatus for determining a femoral neck axis centerline which is configured to guide a rod along said femoral neck axis centerline.

An aspect of the invention comprises a surgical instrument which serves to locate a center axis of a femoral neck by first locating the center of the femoral neck in a fixed first plane. The instrument is then translated and rotated within said fixed first plane to find the center of the femoral neck in a second plane different from the first plane. The intersection of the first and second planes represents a center axis through the femoral neck. Once the center axis of the neck is found, a guide wire is placed within guide means located on the instrument, and the guide wire is inserted into femoral bone. The guide means is configured to locate and position the guide wire along the center axis of the femoral neck. Once the guide wire is inserted into femoral bone, the instrument is then removed while leaving the guide wire in situ.

According to another aspect of the invention a pin alignment guide may be placed on an end of a bone for guiding a pin along a central axis of the bone. The bone has a length extending away from the end of the bone. A body may be configured to overlie the end of the bone. The body may have a contact surface configured to contact the bone and a guide surface configured to guide a pin into the bone. A rod portion may be configured to extend along the length of the bone away from the end of the bone. An extension may be configured to attach to the body and extend away from the body, the extension further configured to couple to the rod portion, such that the rod portion may be aligned along the length of the bone thereby positioning the guide surface of the body to guide the pin.

Another aspect of the invention may include a second extension and a second rod portion. The second extension may be configured to extend from the body in a second direction non-parallel to the direction of the extension. The rod portion and the second rod portion may then extend along the bone in separate planes.

In yet another embodiment, the extension and the second extension may be generally perpendicular to one another.

In another embodiment, the bone is the proximal end of the femur such that the end of the bone is the femoral head and the bone extending from the end of the bone is the neck of the femur.

Another embodiment of the invention provides for the rod portion to align along the central axis of the bone when the pin alignment guide is properly positioned on the end of the bone.

In yet another embodiment, the rod portion includes a generally planar surface configured so as to generally align a plane through the central axis of the bone when the pin alignment guide is properly positioned on the end of the bone.

In another embodiment, the guide surface is a port extending through the body of the pin alignment guide.

Yet another embodiment provides the contact surface to include a generally concave flange to extend over the end of the bone.

Another embodiment provides for the concave flange to include securing means to provisionally secure the pin alignment guide to the bone.

In another embodiment, the guide surface extends away from the body and the end of the bone such that the guide surface aligns axially along the central axis of the bone.

In yet another embodiment, the extension is integrally formed with the body portion.

Another embodiment provides the rod portion is moveably attached to the extension such that the length of the rod extending along the length of the bone may be adjusted.

Yet another embodiment provides a method for placing a pin through an end of a bone and along a central axis of the bone. The bone has a length extending away from the end of the bone. A step orients a first rod portion generally parallel with the central axis of the bone along the length of the bone away from the end of the bone. The first rod portion positions a pin guide body portion over the end of the bone. Another step orients a second rod portion generally parallel with the central axis of the bone along the length of the bone away from the end of the bone. The second rod portion positions the pin guide body portion over the end of the bone. The first and second rod portions, being properly aligned along the length of the bone, orient a pin guide surface in the pin guide body portion such that the guide surface is configured to direct a pin along the central axis of the bone.

Further areas of applicability of the present invention will become apparent from the detailed description provided hereinafter. It should be understood that the detailed description and specific examples, while indicating certain embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is an isometric view of a surgical instrument according to one embodiment;

FIG. 2 is an alternative view of the surgical instrument shown in FIG. 1;

FIG. 3 is a top plan view of the surgical instrument shown in FIGS. 1 and 2;

FIG. 4 is a side plan view of the surgical instrument shown in FIGS. 1-3;

FIG. 5 is an isometric view of a body portion of the surgical instrument shown in FIGS. 1-4; and

FIG. 6 is an isometric view of a rod portion of the surgical instrument shown in FIGS. 1-5.

### DETAILED DESCRIPTION OF THE EMBODIMENTS

The following description of the preferred embodiment(s) is merely exemplary in nature and is in no way intended to limit the invention, its application, or uses.

The invention provides, in part, a method and apparatus for locating and positioning a guide wire within a femoral neck along the femoral neck axis. After the guide wire is positioned and inserted along the femoral neck axis, it is over-drilled to a predetermined depth in order to create a cavity for a stem of a prosthetic femoral head resurfacing implant.

It is preferred that cementless options be utilized, however, acetabular implants of the present invention may incorporate cemented options.

The usefulness of the present invention is not limited to femoral head resurfacing, but may also have some practicality in shoulder arthroplasty (e.g., humeral head resurfacing), knee arthroplasty (e.g., locating the anatomical axis of femur or tibia), or ankle arthroplasty (e.g., finding the proper orientation for talus preparation).

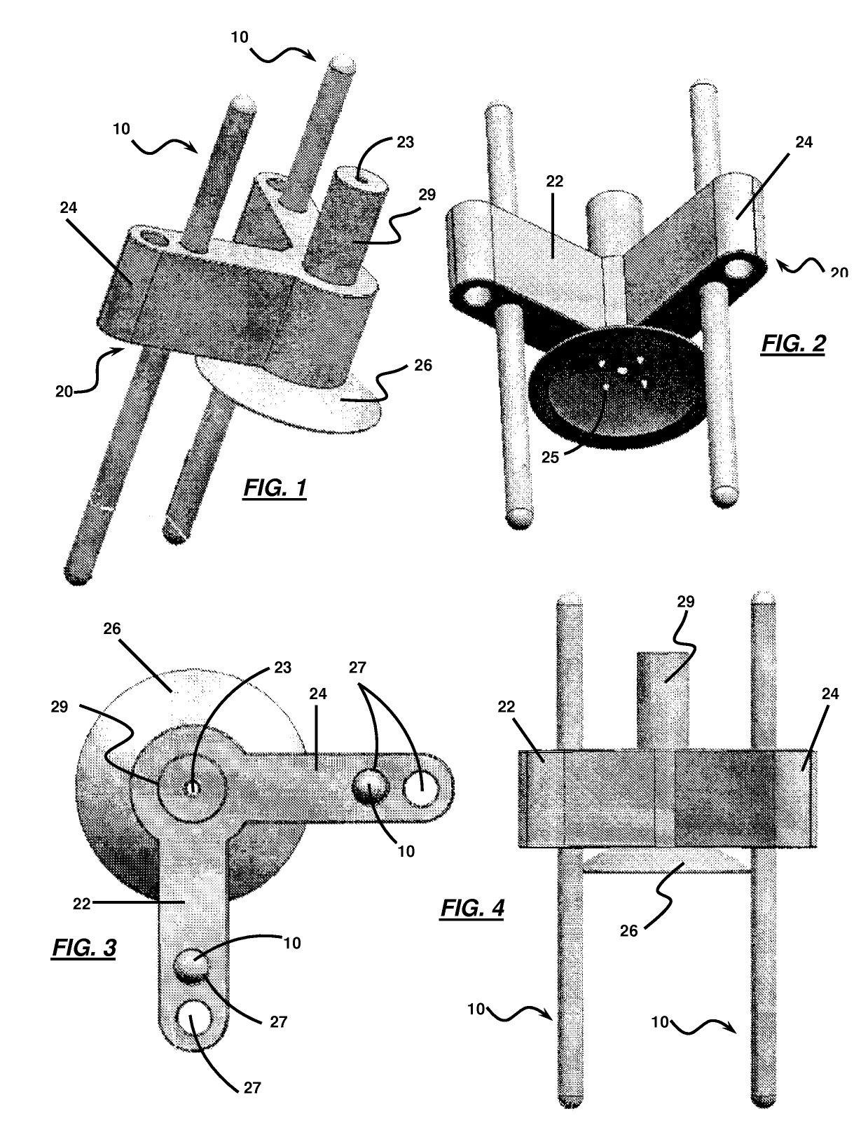

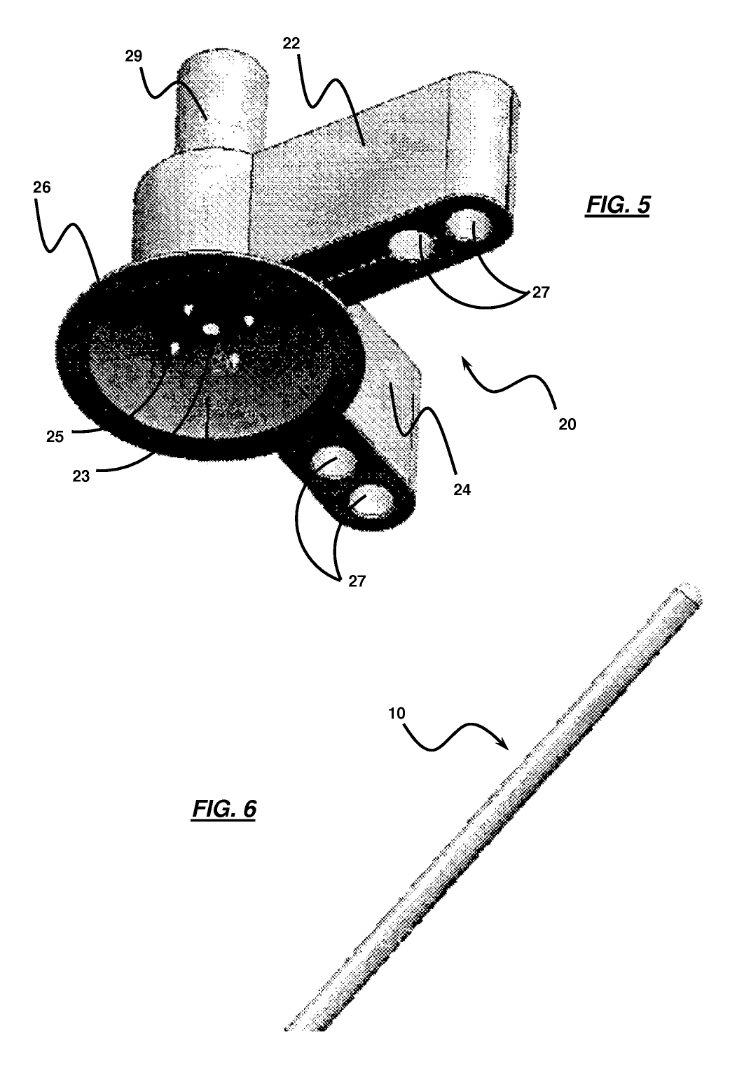

FIG. 1 illustrates a surgical instrument comprising a body portion 20 and one or more rod portions 10. The body portion 20 comprises a first extension portion 22 and a second extension portion 24. Each extension portion comprises rod attachment means which may comprise one or more apertures 27 for receiving one or more rod portions 10. Apertures 27 may be of any size or shape. For example, the apertures 27 may comprise thin rectangular slots, grooves, triangular apertures, or other means for attaching one or more rod portions 10. Rod portions 10 may have any cross-sectional shape so long as they are configured to attach to the extension portions 22, 24. For example, rod portions 10 may be plate-like to better visualize the first and second imaginary planes defined by the rod portions 10 and extension members. In some embodiments where minimally-invasive techniques are undesirable or unnecessary, it may be desirable to make rod portions 10 integral and monolithic with the body portion 20.

As shown best in FIG. 3, extension portions 22 and 24 preferably extend perpendicular with respect to each other. The rod portions 10, when inserted into one or more of the apertures 27 in each of the extension portions, generally form a plane with their respective extension portion. In other words, extension portion 22 and a rod portion inserted in an aperture 27 located on the extension portion 22 will both lie in an imaginary fixed first plane; and extension portion 24 and a rod portion inserted in an aperture 27 located on the extension portion 24 will both lie in an imaginary fixed second plane. Guide means 23 for guiding a guide wire is located along a perpendicular intersection of the first and second planes. While guide means 23 is illustrated as a through-hole, other means for guiding a guide wire are envisaged. For instance, guide means 23 may comprise a slot, slit, or notch. A boss 29 may be provided in order to improve the efficiency of guide means 23 and improve the accuracy of final guide wire placement.

Body portion 20 may comprise a lower flange portion 26 for resting against the femoral head surface. Flange portion 26 may have many different geometries, but preferably has a geometry that increases surface contact with the femoral head. The flange generally serves to increase contact area with the femoral head, without making the entire body 20 large and bulky. Accordingly, the surgical instrument may be advantageously used with smaller incision procedures in a minimally-invasive manner. In some embodiments, the flange may comprise a cup-shaped or generally concave surface for better contact with the femoral head.

Portions of the body 20 or flange 26 that are configured to contact the femoral head may be provided with securing means 25 such as friction-increasing surfaces, spikes (shown in drawings), rough coatings, grit blast surfaces, bumps, protrusions, high friction surfaces, or the like. Securing means 25 ensures little movement of the surgical instrument during guide wire insertion.

In use, the surgeon places the flange portion 26 of the body 20 on the femoral head and then aligns one rod portion 10 and one extension portion 22 such that the rod portion 10 visually tracks down along the center of the femoral neck when viewed in a first direction. This sets the surgical instrument in an imaginary first fixed plane. Once the surgical instrument is established in this imaginary first fixed plane, the surgeon then aligns the other rod portion 10 and the other extension portion 24 such that said other rod portion 10 visually tracks down along the femoral neck when viewed in a second direction perpendicular to the first direction. This essentially locates the guide means 23 so that it is generally co-linear with the center axis of the femoral neck. When the instrument is properly positioned, the securing means 25, such as spikes or teeth within the concave surface of the flange portion 26, engage with the femoral bone to hold the surgical instrument in place relative to the bone for guide wire placement. The surgeon then inserts the guide wire using the guide means 23 as conventionally done.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above described exemplary embodiments, but should be defined only in accordance with the following claims appended hereto and their equivalents.

What is claimed is:

1. A pin alignment guide placed on an end of a bone for guiding a pin along a central axis of the bone, the bone having a length extending away from the end of the bone, comprising: a body configured to overlie the end of the bone, the body having a contact surface configured to contact the bone and a guide surface configured to guide a pin into the bone; a rod portion configured to extend along the length of the bone away from the end of the bone; and an extension configured to attach to the body and extend away from the body, the extension further configured to couple to the rod portion, such that the rod portion may be aligned along the length of the bone thereby positioning the guide surface of the body to guide the pin.

2. The pin alignment guide of claim 1, further comprising a second extension and a second rod portion, the second extension configured to extend from the body in a second direction non-parallel to the direction of the extension, the rod portion and the second rod portion then extending along the bone in separate planes.

3. The pin alignment guide of claim 2, wherein the extension and the second extension are generally perpendicular to one another.

4. The pin alignment guide of any of the above claims, wherein the bone is the proximal end of the femur such that the end of the bone is the femoral head and the bone extending from the end of the bone is the neck of the femur.

5. The pin alignment guide of claim 1, wherein the rod portion aligns along the central axis of the bone when the pin alignment guide is properly positioned on the end of the bone.

6. The pin alignment guide of claim 1, wherein the rod portion includes a generally planar surface configured so as to generally align a plane through the central axis of the bone when the pin alignment guide is properly positioned on the end of the bone.

7. The pin alignment guide of claim 1, wherein the guide surface is a port extending through the body of the pin alignment guide.

8. The pin alignment guide of claim 1, wherein the contact surface includes a generally concave flange to extend over the end of the bone.

9. The pin alignment guide of claim 8, wherein the concave flange includes securing means to provisionally secure the pin alignment guide to the bone.

10. The pin alignment guide of claim 1, wherein the guide surface extends away from the body and the end of the bone such that the guide surface aligns axially along the central axis of the bone.

11. The pin alignment guide of claim 1, wherein the extension is integrally formed with the body portion.

12. The pin alignment guide of claim 1, wherein the rod portion is moveably attached to the extension such that the length of the rod extending along the length of the bone may be adjusted.

13. A method for placing a pin through an end of a bone and along a central axis of the bone, the bone having a length extending away from the end of the bone, comprising: orienting a first rod portion generally parallel with the central axis of the bone along the length of the bone away from the end of the bone, such that the first rod portion positions a pin guide body portion over the end of the bone; and orienting a second rod portion generally parallel with the central axis of the bone along the length of the bone away from the end of the bone, such that the second rod portion positions the pin guide body portion over the end of the bone, the first and second rod portions, being properly aligned along the length of the bone, orient a pin guide surface in the pin guide body portion such that the guide surface is configured to direct a pin along the central axis of the bone.

14. The method of claim 13, wherein the bone is a femoral head of a patient's hip joint such that the pin guide body partner is generally co-linear with the center axis of the femoral neck.

15. A pin alignment guide placed on an end of a bone for guiding a pin along a central axis of the bone, the bone having a length extending away from the end of the bone, comprising: a body configured to overlie the end of the bone, the body having a contact surface configured to contact the bone and a guide surface configured to guide a pin into the bone; a first rod portion configured to extend along the length of the bone away from the end of the bone, and a second rod portion extending along the bone in a separate plane from the first rod portions; and a first extension configured to attach to the body and extend away from the body, the extension further configured to couple to the rod portion, such that the rod potion may be aligned along the length of the bone thereby positioning the guide surface of the body to guide the pin, and a second extension configured to extend from the body in a second direction non-parallel to the direction of the first extension.

16. The pin alignment guide of claim 15, wherein the first extension and the second extension are generally perpendicular to one another.

17. The pin alignment guide of claim 16, wherein the bone is the proximal end of the femur such that the end of the bone is a femoral head and the bone extending from the end of the bone is the neck of the femur.

18. The pin alignment guide of claim 17, wherein the rod portion aligns along the central axis of the bone when the pin alignment guide is properly positioned on the end of the bone.

19. The pin alignment guide of claim 18, wherein the rod portion includes a generally planar surface configured so as to generally align a plane through the central axis of the bone when the pin alignment guide is properly positioned on the end of the bone.

20. The pin alignment guide of claim 19, wherein the guide surface is a port extending through the body of the pin alignment guide.
